(19) United States
(12) Patent Application Publication
(10) Pub. No.: US 2023/0211038 A1
(43) Pub. Date: Jul. 6, 2023

(54) PORTABLE SANITIZER FOR AIRBORN PATHOGENS AND METHOD OF OPERATION THEREOF

(71) Applicant: KONINKLIJKE PHILIPS N.V., EINDHOVEN (NL)

(72) Inventors: SEAN ELWELL, PITTSBURGH, PA (US); ADAM SEIVER, LOS ALTOS HILLS, CA (US)

(21) Appl. No.: 17/988,802

(22) Filed: Nov. 17, 2022

Related U.S. Application Data
(60) Provisional application No. 63/294,893, filed on Dec. 30, 2021.

(51) Int. Cl. A61L 9/20

(52) U.S. Cl. CPC A61L 9/20; A61L 2209/134; A61L 2209/15; A61L 2209/111

(57) ABSTRACT

A hood for a sterilization system including a return line (132) to provide for airflow between first and second ends; a hood (116) having an opening situated in a top wall and configured to be coupled to a tube; and couplers (126) configured to couple the hood to adjacent ones of a plurality of support struts (114) to configure the hood to form an opening (128) leading to a cavity (129), wherein the cavity is configured to receive at least a portion of a head of a subject (101) receiving respiratory gas from a ventilator (170) such that the plurality of support struts are situated outside of the cavity, wherein the hood is configured to enable ambient air into the airflow while over the portion of the head of the subject.

### CROSS REFERENCE TO RELATED APPLICATIONS

This application claims the benefit of U.S. Provisional Patent Application No. 63/294,893 filed Dec. 30, 2021. This application is hereby incorporated by reference herein.

### FIELD OF THE PRESENT SYSTEM

The present system relates to a system to protect a user from airborne pathogens and potentially protect others from pathogens exhaled by the user.

### BACKGROUND OF THE PRESENT SYSTEM

It is too often the case that patients with respiratory disease incidentally contaminate the setting of their treatment and their caregivers due to lack of availability of low cost, portable, compact, effective airflow sterilization measures.

It is also the case that the respiratory devices, such as ventilators, respirators, positive airway pressure (PAP), continuous positive airway pressure (CPAP) devices, and the like, used to serve patients suffering acute respiratory disease become internally contaminated with airborne contaminants when drawing ambient air from the vicinity of the patient's treatment, such as from the same room. This contamination may then be transmitted into the patient's lungs and airways. High-efficiency particulate absorbing (HEPA) and other fine particulate filters are inadequate to guarantee complete sterility of the interior parts of respirators such as PAP devices. HEPA filters also must be frequently replaced to be effective and are prone to performance degradation due to humidity such as may be contained in exhaled air. Further, if HEPA filters are not kept dry or changed frequently enough, contagion may be allowed to escape and airflow pressure may not be properly maintained as filters become clogged or obstructed. Because of contamination, respiratory equipment, including the respiratory equipment inner parts, must be frequently cleaned which is difficult, time consuming, and can cause damage and degradation to the hardware of the respiratory equipment.

Further, conventional respiratory equipment such as ventilators, respirators, PAP machines, CPAP machines, and the like may not sufficiently filter intake air which may lead to secondary infections in subjects receiving respiratory care.

Ventilators, such as PAP devices and the like, may be used in various settings ranging from home to acute care and surgery and are often found everywhere from home residences to enterprises such as health systems, and federal and state emergency response organizations. In these settings, it is difficult to efficiently clean these ventilators in a timely and efficient manner especially in times of urgency.

To overcome the aforementioned barriers and detriments as well as others, there is a need for a sterilization system and method of operation thereof that can effectively filter exhaled gasses and droplets from a patient that may or may not be receiving respiratory assistance such as from a ventilator, a respirator, a PAP machine, and/or a CPAP machine.

### SUMMARY OF THE PRESENT SYSTEM

The system(s), device(s), method(s), arrangements(s), interface(s), computer program(s), processes, etc., (hereinafter each of which will be referred to as system, unless the context indicates otherwise), described herein address problems in prior art systems.

In accordance with embodiments of the present system, there is disclosed a sterilization system for a ventilator return air flow, including: a hood base having an opening and a coupler configured to receive a return line and a plurality of support struts extending away from the hood base; a hood having a top wall and at least one side wall extending from a top wall, the hood being configured to be coupled to the plurality of support struts to form an opening leading to a cavity, the cavity being configured to receive at least a portion of a head of a subject receiving respiratory gas from an air source via a mask; a sterilizer coupled to the return line and having at least one sterilization chamber and a sterilization source configured to emit radiation to sterilize air within the at least one chamber; and an air pump configured to establish an air flow through the opening of the hood and into the at least one sterilization chamber for sterilization to form a sterilized air flow that may be provided to an inlet port of the air source. The hood may be configured to enable ambient air into the airflow while over the portion of the head of the subject. The sterilization system may include a pole stand including a hood support coupled to the hood base. The pole stand may be configured to selectively position the hood in a vertical or a horizontal position. The pole stand may include a base having a plurality of wheels and a support pole which extends from the base. The pole stand may be configured to be coupled to at least one of the sterilizer and the air source.

The sterilization source may include an ultra-violet (UV) lamp configured to emit radiation that is germicidal into the at least one sterilization chamber of the sterilizer. The emitted radiation may have a wavelength between 100-320 nm. The radiation source may include at least one light-emitting diode (LED). The sterilization system may include at least one controller configured to control the intensity of the sterilization source and speed of the air pump such that increasing the speed of the air pump increases the intensity of the sterilization source and decreasing the speed of the air pump decreases the intensity of the sterilization source.

In accordance with embodiments of the present system, there is disclosed a sterilization system for a ventilator return air flow, the sterilization system including: a return line configured to provide for airflow between first and second ends; a hood having a top wall, at least one side wall extending from a top wall, and an opening situated in the top wall, wherein the hood may be configured to be coupled to a tube that is coupled to an end of the return line; and couplers configured to couple the at least one side wall of the hood to adjacent ones of a plurality of support struts to configure the hood to form an opening leading to a cavity, the cavity being configured to receive at least a portion of a head of a subject receiving respiratory gas from an air source via a mask, wherein the hood is configured to enable ambient air into the airflow while over the portion of the head of the subject. The sterilization system may include a hood base configured to be coupled to the plurality of support struts such that each of the support struts extends away from the hood base and each other, the hood base having an opening configured to receive the tube that is coupled to the end of the return line.

The sterilization system may include a sterilizer having at least one sterilization chamber coupled to the return line. The sterilizer may be configured to receive the airflow, sterilize the airflow, and output the sterilized airflow via an outlet port to be provided to an inlet port of an air source. The sterilization system may include at least one sterilization source situated within the at least one sterilization chamber. The at least one sterilization source may be configured to emit radiation to sterilize the air flow within the at least one sterilization chamber. The sterilization system may include a sterilizer output coupler configured to provide the sterilized air flow to the inlet port of the ventilator. The sterilizer output coupler may have a balance valve to selectively bleed at least a portion of the sterilized air flow to an ambient surrounding. The balance valve may be configured to be manually adjusted. The at least one sterilization source may include at least one ultra-violet (UV) light emitting diode (LED). The LED may be configured to emit germicidal radiation into the at least one sterilization chamber of the sterilizer. The sterilization system may include at least one controller configured to control an intensity of the at least one sterilization source. The sterilization system may include an air pump configured to establish the airflow to flow air through the opening of the hood and into the at least one sterilization chamber for exposure to the at least one sterilization source and subsequent output as the sterilized air flow to an inlet port of the ventilator. The sterilization system may include at least one controller to control the air pump to establish air flow through the opening of the hood.

In accordance with embodiments of the present system, there is disclosed a hood for a sterilization system, including a return line to provide for airflow between first and second ends; a hood having a top wall, at least one side wall extending from a top wall, and an opening situated in the top wall and configured to be coupled to a tube coupled to an end of the return line; and couplers configured to couple the at least one side wall of the hood to adjacent ones of a plurality of support struts to configure the hood to form an opening leading to a cavity configured to receive at least a portion of a head of a subject receiving respiratory gas from a ventilator via a mask such that the plurality of support struts are situated outside of the cavity, wherein the hood is configured to enable ambient air into the airflow while over the portion of the head of the subject. The hood may include a sensor configured to sense a CO₂ level within the airflow and to provide sensor information. The hood may include a controller coupled to the sensor. The controller may be configured to reduce the CO₂ level within the airflow.

### BRIEF DESCRIPTION OF THE DRAWINGS

It should be expressly understood that the drawings are included for illustrative purposes and do not represent the scope of the present system. It is to be understood that the figures may not be drawn to scale. Further, the relation between objects in a figure may not be to scale and may in fact have a reverse relationship as to size. The figures are intended to bring understanding and clarity to the structure of each object shown, and thus, some features may be exaggerated in order to illustrate a specific feature of a structure. In the accompanying drawings, like reference numbers in different drawings may designate identical or similar elements, portions of similar elements and/or elements with similar functionality. The present system is explained in further detail, and by way of example, with reference to the accompanying drawings which show features of various exemplary embodiments that may be combinable and/or severable wherein:

FIG. 1 is an illustration of a schematic block diagram which shows a portion of a system operating in accordance with embodiments of the present system;

FIG. 2 is an exploded view of a portion of a system including a patient in accordance with embodiments of the present system;

FIG. 3 is an illustration of a system with the patient in position relative to the hood during use in accordance with embodiments of the present system;

FIG. 4 is a bottom view illustration of a portion of the hood of the system in accordance with embodiments of the present system;

FIG. 5 is a top view illustration of a portion of the hood of the system in accordance with embodiments of the present system;

FIG. 6. is a partially cutaway and exploded side view of the portion of the top wall of the hood in accordance with embodiments of the present system;

FIG. 7 is a partially exploded top view which shows a portion of the base in accordance with embodiments of the present system;

FIG. 8 is a partially-cutaway schematic front view illustration of a portion of the sanitizer in accordance with embodiments of the present system;

FIG. 9 is a partially-cutaway schematic side view illustration of a portion of the sanitizer in accordance with embodiments of the present system; and

FIG. 10 shows a block diagram of a portion of a system in accordance with embodiments of the present system.

### DETAILED DESCRIPTION OF THE PRESENT SYSTEM

The following are descriptions of illustrative embodiments that when taken in conjunction with the following drawings will demonstrate the above noted features and advantages, as well as further ones. In the following description, for purposes of explanation rather than limitation, illustrative details are set forth such as architecture, interfaces, techniques, element attributes, etc. However, it will be apparent to those of ordinary skill in the art that other embodiments that depart from these details would still be understood to be within the scope of the appended claims. Moreover, for the purpose of clarity, detailed descriptions of well-known devices, circuits, tools, techniques, and methods are omitted so as not to obscure the description of the present system.

The term “and/or,” and formatives thereof, should be understood to mean that only one or more of: the recited elements may need to be suitably present (e.g., only one recited element is present, two of the recited elements may be present, etc., up to all of the recited elements may be present) in a system in accordance with the claims recitation and in accordance with one or more embodiments of the present system. In the context of the present embodiments, the terms “about”, substantially and “approximately” denote an interval of accuracy that a person skilled in the art will understand to still ensure the technical effect of the feature in question which in some cases may also denote “within engineering tolerances.” The term may indicate a deviation from the indicated numerical value of ±20%, ±15%, ±10%, ±5%, ±1%±0.5% or ±0.1%.

The terms user, users, or formatives thereof may refer to a user, operator, clinician, technician, and/or the like unless the context indicates otherwise.

The terms patient, patients, or formatives thereof, may include patients or other individuals (e.g., subjects, etc.) utilizing the present system and may include a party receiving any type of breathable airflow included as used for respiratory assistance, therapy, and/or the like from respiratory device such as a respirator, a ventilator, a positive airway pressure (PAP) machine, a continuous PAP (CPAP) machine and/or the like. Though a ventilator is illustratively described in the following to simplify the discussion, however, it should be appreciated that any source of air suitable for being provided to a patient may be readily utilized. Therefore, the term ventilator herein should be understood to include, unless the context herein indicates otherwise, any suitable source including a breathing therapy device, such as a medical device for diagnosing or treating respiratory conditions such as a positive airway pressure device (e.g., bi-level positive airway pressure device, continuous positive airway pressure device, variable positive airway pressure device), oxygen concentrator, air compressor, mechanical insufflation-exsufflation device, resuscitator, ventilator, air pump, or any other device for providing a breathable gas to a patient including for purposes of respiratory treatment or diagnosis. In addition, while a respirator is illustratively described, the present system may be utilized without use of any breathing therapy/treatment/diagnosis device being utilized. For example, the present system may be utilized solely for the purposes of limiting contamination of an ambient environment with contaminants contained in a patient's exhaled airflow.

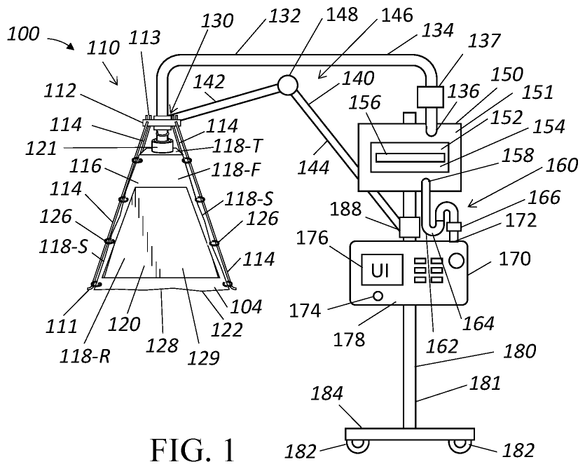

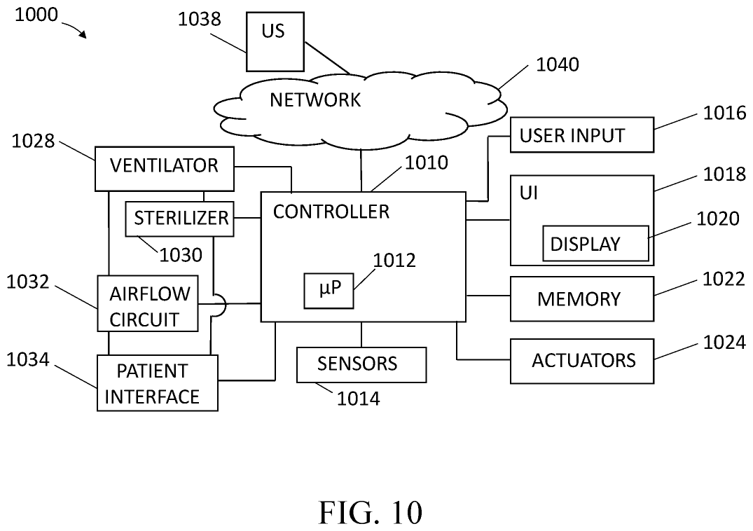

FIG. 1 is an illustration of a schematic block diagram which shows a portion of a system 100 (hereinafter the system 100) operating in accordance with embodiments of the present system. The system 100 may include one or more of: a hood assembly 110, a hood support 146, a return line 132, a sterilizer 150, ventilator 170, a controller, a memory, as illustratively shown in FIG. 10, and a pole stand 180.

The controller may control the overall operation of the system and may include one or more logic devices such as a microprocessor or the like. The controller may access the memory and may obtain system setting information (SSI) which may include one or more of operating parameters (e.g., air pump speed, etc.), threshold values, warnings, display settings, user information, patient information, etc. The SSI may be set by the system and/or user and may be updated during use.

The pole stand 180 may include a base 184 coupled to a support pole 181, and wheels 182. The wheels 182 may be configured to provide mobility to the pole stand 180 during use such that the system 100 may be easily ported within a caregiving space. It is envisioned that the pole stand 180 may be configured to support the hood support 146, the sterilizer 150, the hood assembly 110, and the ventilator 170 during use. In some embodiments, a low-friction material such as Teflon sliders or the like may be provided to provide mobility to the system rather than the wheels 182. In some embodiments, the base 184 may include three or more wheels 182 for stability. In some embodiments, the support pole 181 may telescope to provide for height adjustment by a user, etc.

The hood support 146 may be configured to support the hood assembly 110 in a desired position and/or orientation relative to the support pole 181. The hood support 146 may include first and second support arms 140 and 142, respectively, coupled to each other by a coupler 148 that may provide for one or more degrees of freedom of travel (e.g., linear and/or rotational) between the first and second support arms 140 and 142, respectively. The first support arm 140 may be coupled to the support pole 181 via a coupler 188 configured to provide one or more degrees of freedom of travel (e.g., linearly and rotationally) to the first support arm 140 relative to the support pole 181. For example, the coupler 188 may telescope relative to a longitudinal axis of the support pole 181 to provide for height adjustment and may rotate about the longitudinal axis of the support pole 181 to provide for rotational location adjustment relative to the base 184.

In accordance with embodiments, the first support arm 140 may be configured to be coupled to another support structure (e.g., a structure other than the pole stand 180), such as may be provided on a patient's bed, such as a support bracket. In accordance with embodiments, the first support arm 140 and/or the coupler 188 may provide a mechanism for coupling to other structure, such as a supporting wall. For example, a coupling such as a connector device, connection structure and/or portion thereof as described in U.S. Patent Publication No. 2014/0326237, which is incorporated herein by reference as if set out in entirety herein, may be provided as the coupler 188 that enables affixation and monitoring thereof, of the first support arm to a support structure, such as a wall, device, floor, etc., in the vicinity of the patient. As further contemplated, the second support arm 142 may couple directly to the coupler 188 to provide coupling to a support structure (e.g., bed, wall, device, etc., as described) and rotational and/or elevational positioning of the hood 116 without use of the first support arm 140.

The second support arm 142 may include a hood coupler as will be discussed below configured to couple to one or more portions of the hood assembly 110. Locking mechanisms may be provided to lock the couplers in a fixed position when desired and may include any suitable locking mechanism such as a pin, an interference fit, a clamp, etc. By providing one or more degrees of freedom of travel between the hood assembly 110 and the support pole 181, the coupler 188 and/or portions thereof, the hood assembly 110 may be rotated and/or elevated to a vertical position (as shown) to fit over a head of a patient that is walking or sitting, to a horizontal position to fit over a head of a patient that in lying in bed, or any other suitable position or positions as may be desired. In some embodiments, the coupler(s) may provide a plurality of discrete positions of travel such that predetermined positions and/or orientations of the hood assembly 110 may be quickly and easily achieved.

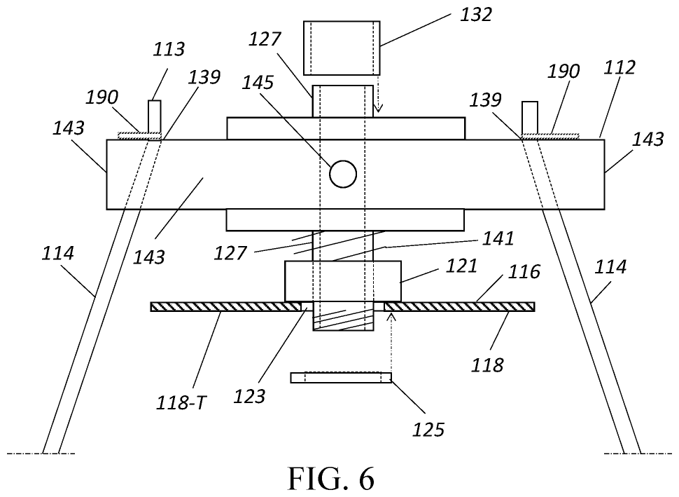

The hood assembly 110 may include one or more of a base 112, support struts 114, a hood 116, a return line coupler 130, hood couplers 126 and one or more strut retainers (hereinafter, retainers, e.g., see, FIG. 6, retainers 190). The support struts 114 may include proximal and distal ends 113, 111, respectively, and may be coupled to the base 112 such that support struts 114 may extend away from the base 112 and away from each other. The support struts 114 may be configured to support the hood 116 in a desired position during use and may be formed from any suitable material such as wood, metal, plastic, a fiber, a composite (e.g., carbon fiber, fiberglass, etc.), etc. For example, it is envisioned that the support struts 114 may be formed from a composite such as carbon fiber or fiberglass such that the support struts 114 may flex when subject to a desired force and may maintain sufficient tension in portions of the hood 116 when installed. The distal end 111 of each support strut 114 may be shaped and/or sized to prevent accidental injury if engaged by a person during use. For example, the distal end 111 of each support strut may be curved over itself or may form a ball to prevent injury during use. In addition, the distal end 111 of the support struts 114 may be contained within a portion of the hood 116, such as within a pocket, sleeve, strap, etc., of the hood 116 when inserted. By extending the support struts 114 away from each other, tension may be maintained across the hood 116 as will be described below in further detail. It should be readily appreciated that in accordance with embodiments, the support struts 114 may not be required as the hood 116 may be formed from a material and/or may include a structure that provides for a shaping to enable the hood 116 to placed over a head of the patient as described herein.

The hood 116 may include at least one wall such as a top wall 118-T and front and rear walls 118-F and 118-R, respectively, which may extend between side walls 118-S, all generally referred to as “at least one wall 118”. Although illustratively, the top wall 118-T is shown positioned away from the base 112, it is envisioned that the top wall 118-T may be positioned adjacent (e.g., contiguous with or immediately adjacent) to the base 112. The at least one wall 118 may include an edge 122 defining an opening 128 leading to a cavity 129. The at least one wall 118 may include a hem 104 leading to the edge 122. The hem 104 may be longer or shorter to provide material (e.g., flexible material) that may act as draping material that may extend from a supported portion of the hood 116 to conform to a portion of the patient, such as the patient's body below their head, to facilitate a reduction of airflow into the cavity 129 from the opening 128 and the ambient environment. It is envisioned that although the hood 116 is illustratively shown as being symmetrical along the edge 122, the edge 122 may be asymmetrical such that the hood 116 is shorter or longer along one or more dimensions extending from the top wall 118-T to the edge 122, for example to more readily accommodate a positioning, such as when the hood 116 is positioned over a reclining patient, a front of the hood may be longer than a back of the hood (e.g., with the front and back of the hood corresponding to a front and back of the patient). In other embodiments, the back of the hood may be longer than the front of the hood. The at least one wall 118 may include at least one window such as a window 120 situated in the front wall 118-F. The at least one window may be formed from any suitable material which may be flexible. The window 120 may include any suitable clear panel that may provide visibility into and out of the hood 116 when placed over the head of a patient in use. In some embodiments, portions of the hood 116 may be formed from a flexible material such as paper, plastic, etc. and may be fully disposable. The rear wall 118-R and cavity 129 are seen through the window 120. For example, it is envisioned that the hood 116 may be constructed from recyclable and/or compositable material. The top wall 118-T may be positioned against, or adjacent to a flange 121 to maintain a seal.

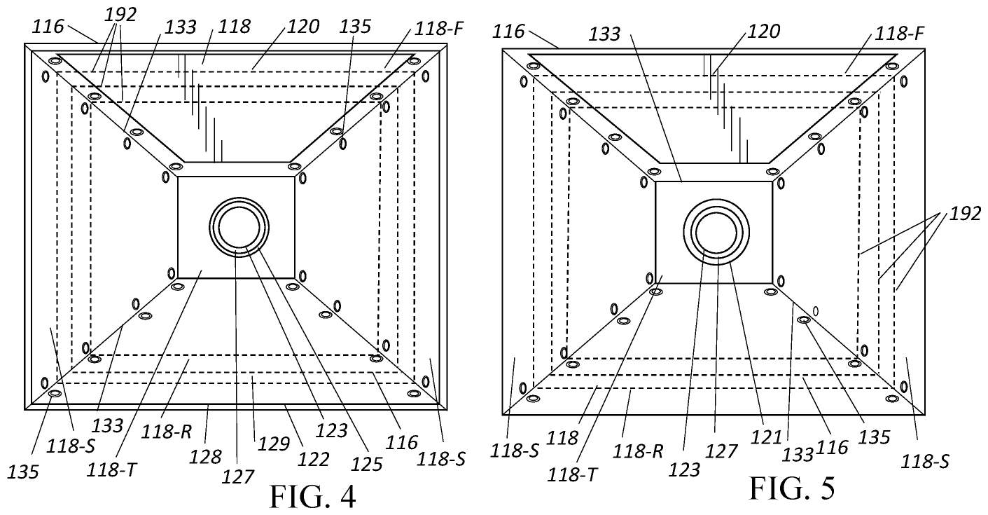

The couplers 126 may be configured to secure the hood 116 to the support struts 114 in embodiments when the support struts are present. The couplers 126 may include any suitable coupler or fastener (e.g., spring clips, staples, rings, etc.) which may extend around a corresponding support strut 114 and through adjacent attachment openings (e.g., see, FIG. 4, openings 135) in the at least one wall 118. As readily appreciated, the openings 135 and/or other support structure of the hood 116 is fluidically sealed from the cavity 129 and as such for example, does not provide a way for air entrance or egress to/from the cavity 129. In this way, it is contemplated that a negative pressure airflow may be created around the patient when the hood 116 is in use without an entrance or egress of airflow through the openings 135 and/or other support structure of the hood 116.

In some embodiments, it is envisioned that the fasteners 126 may be formed from a biasing material such as a spring clip or ring that may be easily manipulated by a user when coupling the at least one wall 118 to the struts 114. In other embodiments, the at least one wall 118 may be formed to include one or more of the fasteners 126 (e.g., formed as a pocket, sleeve, strap, etc.) around corresponding support struts 114. It is envisioned that the openings 135, when present, may be reinforced using any suitable reinforcement such as grommet, a ring, webbing, etc., which may be secured to the at least one wall 118. The fasteners 126 may transfer a tension from the one or more support struts 114 across the corresponding walls (e.g., 118-F, 118-R, 118-S) of the hood 116 to maintain a desired shape and/or form of the hood 116 and the cavity 129 within. In this way, an outflow of air through the opening 128 from within the cavity 129 may be prevented during use. Accordingly, in some embodiments, the one or more support struts 114 may be shaped and/or sized sufficiently and/or may be formed from a suitable material (e.g., flexible fiberglass, carbon fiber, etc.), to provide a sufficient biasing force as may be desired across the corresponding walls (e.g., 118-F, 118-R, 118-S) of the hood 116. In some embodiments, the walls 118 may include a biasing member (e.g., a spring, a biasing band, material, the fasteners 126, etc.) to provide for a desired amount of bias across one or more corresponding walls (e.g., 118-F, 118-R, 118-S) of the hood 116.

The return line coupler 130 may be configured to couple a distal end of the return line 132 to the hood assembly 110 such that the cavity 129 of the hood assembly 110 may be flow coupled to the return line 132. The return line 132 may include a flexible hose 134 or the like which may flow couple the cavity 129 to the sterilizer 150. In some embodiments, the flexible hose 134 may be disposable. It is further envisioned that the return line 132 may include a filter (e.g., see, element 137 described further herein) configured to filter debris of a desired size from airflow through the return line 132 which airflow may be provided to the sterilizer 150. The filter, such as a HEPA filter, may be configured to screen out fine particulates or the like incidentally carried in the air flow. In some embodiments, one or more sensors (e.g., see, element 137 described further herein) may be positioned and/or configured to sense characteristics of the gasses within portions of the system 100, such as within the cavity 129, within the return line 132, within the sterilizer 150, etc.

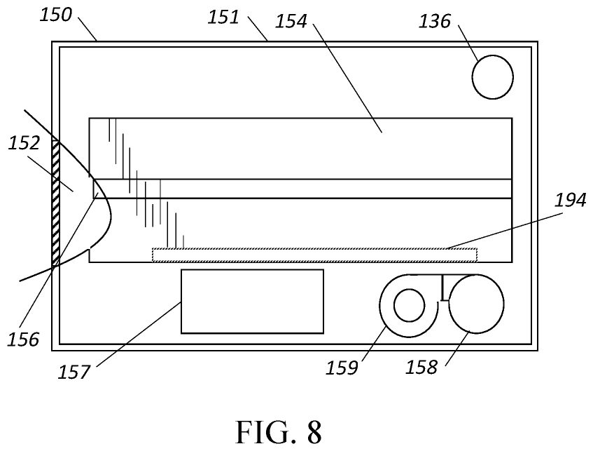

The sterilizer 150 may include one or more of a body 151, at least one sterilization chamber 152, a pump (e.g., see, FIG. 8, blower 159), a controller (e.g., see, FIG. 10, controller 1010), a window 154, a sterilization source (STS) 156, and inlet and outlet ports 136, 158, respectively. The inlet port 136 may be configured to be coupled (e.g., fluidically coupled) to the return line 132 to receive the airflow therefrom. The air flow may then be provided to the at least one sterilization chamber 152 which may be flow situated between the inlet and outlet ports 136, 158, respectively. The STS 156 may include a light source which may include any suitable germicidal lamp or the like that may be configured to illuminate at least a portion of the at least one sterilization chamber 152 with an ultra-violet (UV) light having a desired wavelength or wavelengths (e.g., a germicidal wavelength or range of wavelengths, such as between about 100-320 nm) such that the emitted illumination may lie in the UV-C spectrum (e.g., 100-280 nm). In some embodiments, the illumination may have a wavelength of between 220-260 nm. However, other wavelengths or ranges of wavelengths are also envisioned. In some embodiments, it is envisioned that the STS 156 may include UV-C light emitting diodes (LEDs) and may be tunable in wavelength and/or frequency. It is envisioned that the STS 156 may be controlled by the controller (e.g., see, FIG. 10, controller 1010) which may control the wavelength and/or intensity of the emitted light in accordance with system and/or user settings. For example, in some embodiments, when fan speed is increased, the system/controller may control the LEDs to increase an intensity of the emitted illumination. Conversely, when the fan speed is decreased, the system/controller may control the LEDs to decrease the intensity of the emitted illumination (e.g., in accordance with system and/or user settings). In accordance with embodiments of the present system, it is also envisioned that the wavelength and/or intensity of the emitted light may be preset, such as by a selecting a given STS 156 with desired operating characteristics. It is envisioned that the STS 156 may further include an X-ray and/or gamma sources that may emit x-rays and/or gamma rays, respectively, or other wavelengths within the electromagnetic spectrum that may destroy or incapacitate airborne pathogens within the at least one sterilization chamber 152 as may be desired. In some embodiments the STS 156 may be tunable by the controller. Adequate shielding of the sanitizer 150 may be provided depending upon type of disinfection methods (e.g., UV-C, X-ray, etc.) employed by the STS 156.

An air pump (e.g., see, FIG. 8, blower 159) may be flow coupled to the at least one sterilization chamber 152 and may be configured to pump gasses from/to the at least one sterilization chamber 152 via the inlet and outlet ports 136, 158, respectively, to exhaust gasses from the at least one sterilization chamber 152. The operation of the pump may result in a vacuum (e.g., reduced pressure) within the at least one sterilization chamber 152, the hose 132, and the cavity 129 of the hood 116 relative to ambient pressure.

The outlet port 158 may be flow-coupled to an inlet port 172 of the ventilator 170, when the ventilator 170 is present, via a sterilizer output coupler 160. The sterilizer output coupler 160 may include one or more of a conduit 162, a balance valve 166, a trap 164, a filter, a sensor, etc., and may be configured to receive the sterilized flow of air from the outlet port 58 and provide at least some of this airflow to the ventilator 170. The present system in accordance with embodiments may bleed off an excess of the sterilized air flow via the balance valve 166. The conduit 162, or portions thereof, may be formed from any suitable material such as rigid and/or non-rigid tubing, hard or soft plastic tubing, etc., which may be replaceable/disposable or may be fixedly attached to the ventilator 170. The trap 164 may be configured to trap any excess liquid and/or debris from airflow within the conduit 162. The trap 164 may function as a portion of a filter, sensor, etc., as desired.

The balance valve 166 may be configured to bleed excess air (e.g., from the sterilized air flow) from the sterilizer output coupler 160 to prevent excess flow of air from entering the ventilator 170 via the ventilator inlet port 172. The balance valve 166 may be fixed or adjustable and may include a biasing valve which may be configured to control a valve configured to bleed excess air flow when, for example, a pressure threshold between the airflow within the conduit 162 and ambient pressure (e.g., room air pressure) is greater than a threshold value and/or the absolute pressure is greater than a threshold value as may be set by the system and/or user. The balance valve 166 may be fixedly or adjustably set by the user and/or by the system/controller. In some embodiments, an actuator controlled by the controller may be provided to adjust and/or open or close the balance valve 166 under the control of the system/controller. Accordingly, one or more sensors may be provided in the sterilizer output coupler to sense one or more of air speed and pressure, form corresponding air speed and pressure, information, and provide this information to the controller for further processing. The controller may then compare one or more of the air speed and pressure information with corresponding air speed and pressure thresholds, respectively, and may actuate the actuator to open the balance valve 166 when it is determined that one or more of the air speed and pressure information is greater and/or lesser than desired air speed and pressure thresholds. The air speed and/or pressure thresholds may be set by the user and/or system and may be stored in a memory of the system. In some embodiments, the controller may obtain the airspeed and/or pressure information and adjust the STS 156 (e.g., to control light intensity, frequency, etc.) in accordance with actual airspeed/pressure within the system and the airspeed/pressure setting information as may be set by the user and/or system to ensure that airflow exiting the at least one sterilization chamber 152 is sterilized as desired. The airspeed/pressure setting information may be stored in a memory of the system.

The sterilized air that is bled by the balance valve 166 may be referred to as bleed air. Bleeding excess air from the sterilized air flow may be desirable to make up for differences between a volume of air that flows from the sterilizer 150 and a volume of air that may be (or is desired to be) input to the ventilator 170 via its inlet port 172.

It is envisioned that the balance valve 166 may be passively controlled and/or may be actively controlled by the system/controller. Accordingly, the balance valve 166 may allow excess sterilized air to exit a return airflow loop (e.g., prior to entering the ventilator 170) thereby permitting appropriate pressure to be provided (e.g., to/by the ventilator 170 or other return circuit such as a return hose) to the patient for breathing without contaminating an ambient environment around the patient. The controller may control the air pump such that a vacuum (e.g., a pressure below ambient pressure) within the cavity 129 of the hood assembly 110 may be controlled as desired by the system and/or user. For example, a vacuum control selector may be provided (e.g., a knob and/or on a display of the sterilizer, system/controller, etc.) and adjusted by a user, system/controller, etc., to increase the pump speed to temporarily increase the magnitude of a negative pressure zone within portions of the cavity 129 for additional protection while performing close and/or dangerous tasks such as intubation, extubation, airway fluid removal, patient eating and/or drinking, etc. In some embodiments, the vacuum control selector may be realized in hardware (e.g., an adjustable knob situated on the sterilizer 150) or software (e.g., selection items which may be rendered on a rendering device of the system such as on a display 176 of the ventilator 170). However, in yet other embodiments the rendering device may be situated on the sterilizer 150. For example, it is envisioned that the system/controller may generate a graphical user interface (GUI) including an option for a user to select to adjust vacuum selection which may cause the system/controller to control the air pump to increase/decrease (e.g., temporarily or until the setting is changed) the magnitude of a negative pressure zone within portions of the cavity 129.

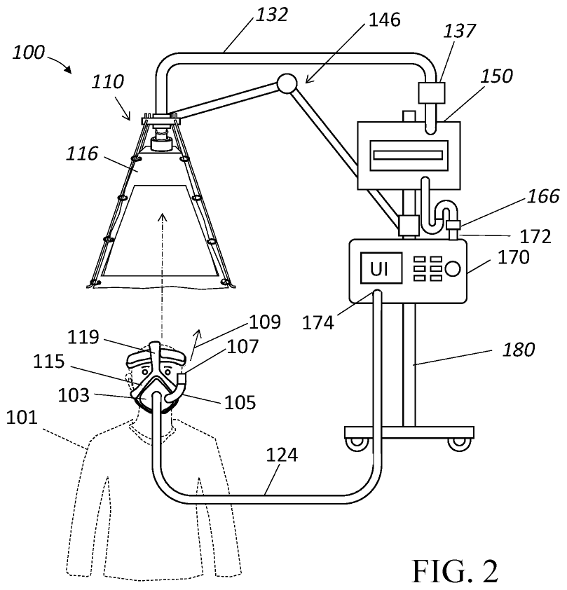

The ventilator 170 or other airflow circuit may receive the airflow from the sterilizer 150 via the inlet port 172 and may further condition this airflow before venting to the ambient environment. The ventilator 170 or other airflow circuit may receive/pump ventilation gas which may include ambient air and/or other gasses (e.g., oxygen (O₂), nitrogen (N₂), etc.), via an outlet port 174 to the patient via an airflow circuit and patient interface such as a mask as will be described with reference to FIG. 2 below.

FIG. 2 is an exploded view of a portion of the system 100 of FIG. 1 or other desired system (e.g., an embodiment) including a patient 101 operating in accordance with embodiments of the present system. The patient 101 may receive the ventilation gas from the sterilizer 150, the ventilator 170, etc., via an airflow circuit 124, and a patient interface 115 coupled thereto, or simply from the airflow circuit 124 itself. To facilitate the discussion, a patient interface is discussed. However, it should be appreciated that the discussion includes embodiments wherein the ventilation gas received by the patient is provided to the patient without a separate patient interface. For example, the ventilation gas may simply be provided to the cavity 129 of the hood 116. In accordance with embodiments, the patient interface 115 may include a mask 103 having a chamber and which may be coupled to the airflow circuit 124 to receive ventilation gas from the output port 174 of the ventilator 170 and provide this ventilation gas to the patient 101 for breathing. Although a mask is illustratively described, other suitable systems may be employed such as a trachea tube, nasal cannula, partial facemasks and others. To simplify the following discussion, a mask is described though should be understood to encompass other suitable patient interfaces.

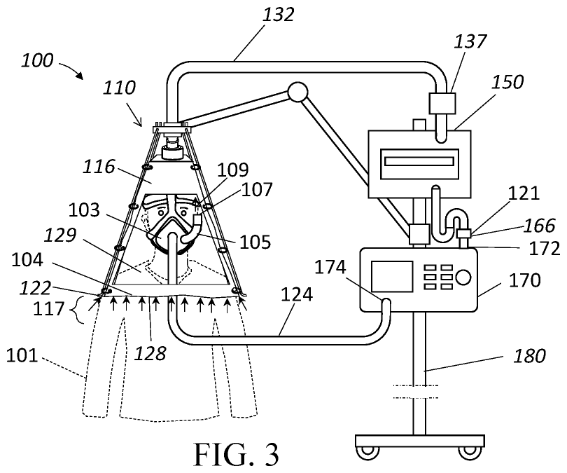

The patient interface 115 may be coupled to the patient 101 via any suitable coupler such as straps 119 which may be configured to maintain the position and/or orientation of at least the mask 103 relative to the face of the patient 101. Exhalation gasses as well as excess gasses from within a chamber of the mask 103 may be output via an opening 107 of an outlet tube 105 coupled to the chamber of the mask 103. A one-way valve may be configured to vent the exhaled gasses from the mask 103 via the outlet tube 105 and prevent inhalation of air through the outlet tube 105 in the opposite direction. The ventilator 170 may maintain positive airway pressure at one or more times within the airflow circuit 124 and/or mask 103. Accordingly, exhaled gasses from the patient 101 may be selectively controlled by the one-way valve for output via the opening 107. These exhaled gasses then may be drawn by a negative pressure maintained within the cavity 129 of the hood 116 as illustrated by arrow 109 and thereafter provided to the sterilizer 150 for sterilization. As previously described, the sterilized air flow may be bled via the balance valve 166 to atmosphere and/or provided to the inlet port 172 of the ventilator 170 for further processing. Thus, all of the air exhaled by the patient 101 via the outlet tube 105 may be drawn into the sterilizer 150 for sterilization prior to being released to the ambient atmosphere or drawn into the ventilator 170 (or otherwise being provided back to the patient). Although typically the patients head is positioned within the cavity of the hood 116 such as shown in FIG. 3, the hood 116 may be positioned above the patient and/or above the patent's mouth during operation, such as to enable the patient to eat/drink. By maintaining a negative pressure within the cavity of the hood 116, airway gases and contaminants (e.g., bacterial and/or viral particles) may be drawn into the cavity and thereby, avoiding contamination of the surrounding environment even when not covering or not completely the patient's head.

FIG. 3 is an illustration of the system 100 of FIG. 1 or other desired system with the patient in position relative to the hood 116 during use in accordance with embodiments of the present system. When the air pump is operating, it may draw air from the hood 116 via the return line 132 and sterilizer 150 which may create a negative pressure region (e.g., relative to ambient pressure) within the cavity 129 of the hood 116. This negative pressure region may gently draw air from the surroundings into the cavity 129 of the hood 116 via the opening 128 as illustrated by arrows 117. As previously described, the hood 116 may include the hem 104 that extends to the edge 122 to facilitate conforming to the patient 101 and a reduction of the amount of ambient air entering the opening 128. In this way, the negative pressure within the hood 116 is more easily provided and/or controlled. As shown, the arrows 117 may indicate a direction of air flow through the opening 128 and into cavity 129 of the hood 116. In accordance with the present system, the negative pressure provided within the cavity 129 may prevent exhaled air from the patient 101 from escaping the cavity 129 prior to sterilization. For example, some of the airflow drawn into the cavity 129, as illustrated by the arrows 117, may pass the opening 107 of the outlet tube 105 thus drawing any air that may be output at the opening 107 of the tube 105 (e.g., exhaled air), as well as any mask leakage, into the sterilizer 150 for sterilization via the return line 132 and the sterilizer 150. This flow may establish at least part of a return airflow loop between the opening 128 of the hood 116 and the ventilator 170 in which excess sterilized airflow may be dumped by the balance valve 166 at an output opening to the atmosphere.

An element 137 may interact with the airflow within the return loop (e.g., such as in the return line 132) and may include one or more of a filter, a CO₂ capture/venting system (e.g., a CO₂ scrubber), a heater, a cooler, a moisture removal/additive system and at least one sensor. The moisture removal/additive system when present (e.g., a dryer) may be configured to dry/moisten the airflow which passes through the return line 132 (hereinafter return airflow) to dry/moisten the return airflow prior to filtration and sampling by the sensors as will be described below. The filter may filter debris of a desired size from the return airflow.

The sensors may sense conditions of the return airflow (e.g., oxygen content, carbon dioxide content, nitrogen content, moisture content, ozone content, airflow, pressure, temperature, etc.), form corresponding sensor information, and provide this sensor information to the controller for further processing in accordance with embodiments of the present system. It is envisioned that the sensors may include one or more of a temperature sensor, a humidistat (e.g., humidity sensor), a carbon dioxide (CO₂) sensor, an oxygen sensor, an airflow sensor, a nitrogen sensor, an ozone sensor, a pressure sensor, etc., each of which may form corresponding sensor information, and provide this information to the controller for further processing. For example, the humidistat when present may sense humidity in the return airflow, form corresponding humidity information and provide this information to the controller for further processing such as to determine humidity within the sensed airflow. Thereafter, the controller may compare the determined humidity with a threshold humidity value (as may be stored in memory of the system), and if it is determined that the humidity is greater/less than the threshold humidity value, the processor may cause this information to be rendered indicating such (e.g., “high humidity sensed,” “replace filter,” etc.) as may be stored in the memory of the system. Further, when the humidity is greater/less than the threshold humidity value, the processor may cause a dryer system (e.g., element 137) to dry/moisten, respectively, the airflow within the system.

Similarly, the CO₂ sensor (e.g., element 137) may sense CO₂ levels within the return airflow, form corresponding CO₂ information and provide this information to the controller for further processing such as to determine a CO₂ concentration within the return airflow Then, the controller may compare the determined CO₂ with a threshold CO₂ value (as may be stored in memory of the system), and if it is determined that the determined CO₂ is greater than the threshold CO₂ value, the processor may cause this information to be rendered indicating such (e.g., “high CO₂ sensed,” “replace filter”, “replace CO₂ scrubber”, etc.) as may be stored in the memory of the system.

The system/controller may control the speed of the air pump to increase/decrease and thus, increase/decrease the return airflow including the air drawn into the cavity 129 from the ambient environment, in accordance with the determined CO₂ levels (e.g., increase the airflow when the CO₂ level is high) as may be set by the system and/or user (e.g., in the SSI). In accordance with embodiments, when the determined CO₂ is greater than the threshold CO₂ value, the controller may cause the CO₂ scrubber/vent to capture/vent excess CO₂ within the airflow (e.g., such as by the controller controlling a valve to divert airflow through the CO₂ scrubber/vent) and/or may control a valve to add 02 into the airflow to thereby, reduce the overall concentration of CO₂ in the airflow and/or to increase the concentration of 02 in the airflow. Further, when the determined CO₂ is greater than the threshold CO₂ value or is greater than some other value, such as a value below the threshold, even when the CO₂ scrubber/vent is operated, thereby indicating that the CO₂ scrubber/vent is not operating at peak CO₂ removal capacity, the controller may render on the user interface a message to replace the CO₂ scrubber/vent when possible and may also render a message to discontinue use of the system when convenient, till replacement of the CO₂ scrubber/vent.

A temperature sensor may sense the temperature of the airflow. The controller may in response control a heater/cooler to heat/cool (e.g., element 137), respectively, the airflow when the airflow temperature is below/above a predetermined threshold value. In this way, the comfort of the patient inhaling the airflow may be improved.

A pressure sensor (e.g., the element 137 positioned as shown and/or positioned within the cavity 129, such as on an inside portion of the hem 104, etc.) may sense the pressure within the cavity 129. The system/controller in response to the sensed pressure may control the speed of the air pump (and the STS 156 as described) to increase/decrease and thus, increase/decrease the return airflow (and sterilization rate) including the air drawn out of the cavity 129, in accordance with determined pressure levels as may be set by the system and/or user (e.g., in the SSI). For example, the system/controller in response to the sensed pressure may increase the airflow drawn out of the hood 116 when the sensed pressure level is not below ambient pressure, is not more than a predetermined amount below the ambient pressure, etc. Further, the system/controller in response to the sensed pressure may decrease the airflow drawn out of the hood 116 when the sensed pressure level is below ambient pressure, is more than the predetermined amount below the ambient pressure, etc. In this way in accordance with embodiments, when the determined pressure inside the cavity 129 is greater (or less) than a threshold pressure value (e.g., such as due to an increase or decrease in the respiration rate of the patient, due to a repositioning of the patient, due to a repositioning of the hood, etc.), the controller may control (e.g., automatically without intervention) the speed of the air pump to increase/decrease and thus, increase/decrease the return airflow including the air drawn out of the cavity 129, in accordance with the determined pressure levels and the desired pressure level as may be set by the system and/or user (e.g., in the SSI). For example, the system/controller may increase the airflow drawn out of the cavity 129 when the sensed pressure level is not more than a predetermined amount, such as 20/40/60 mm of mercury, below the ambient pressure. In accordance with embodiments of the present system, controlling the negative pressure within the cavity 129 helps ensure that no air within the cavity 129 is expelled from the opening 128, thereby ensuring that the ambient environment is not contaminated by the patient's expelled air. As readily appreciated, a further sensor may be provided outside of the cavity 129 so the system/controller may determine the ambient pressure or the ambient pressure may be set by the system and/or a user.

The SSI may include one or more tables which may be accessed to determine system settings such as operating parameters for one or more of the STS 156, the air pump, threshold values, warning codes, default settings, user information, etc. The SSI may be set by the system and/or user and may be stored in a memory (e.g., see, FIG. 10, memory 1022) of the system.

FIG. 4 is a bottom view illustration of a portion of the hood 116 of the system 100 in accordance with embodiments of the present system; FIG. 5 is a top view illustration of a portion of the hood 116 of the system 100 in accordance with embodiments of the present system; and FIG. 6. is a partially cutaway and exploded side view of the portion of the top wall 118-T of the hood 116 in accordance with embodiments of the present system. With reference to FIGS. 4 through 6, the other side views may be similar and are not shown for the sake of clarity. However, the other sides of the hood 116 may be different than those shown, such as when the hood 116 is asymmetrical or is otherwise adjusted to be asymmetrical as discussed.

With reference to FIGS. 4 through 6, the top wall 118-T may include an opening 123 configured to receive a tube 127 and may be sandwiched between the flange 121 and a collar 125 to prevent leakage of air from the opening 123. The flange 121 and the collar 125 may each be coupled to the tube 127 using any suitable method such as threaded coupling, an interference fit, etc. Folds 133 may be located between adjacent ones of the top wall 118-T, the front and rear walls 118-F and 118-R, respectively, and the side walls 118-S of the hood 116. Folds 133 may enable folding of the hood 116. Other folds maybe provided on the at least one wall 118 to facilitate folding of the hood 116 for storage, shipping, and/or to alter a fitting of the hood 116, etc. For example, the hood 116 may include folds 192 to enable an adjustment of the hood 116, such as when the hood 116 is manufactured as a single size (e.g., extra-large), yet through operation of the folds 192, is adjustable to accommodate other sizes, such as small, medium and large. In these embodiments, the hood 116 may be collapsed along one or more of the folds 192 to adjust the size to accommodate different users.

In these embodiments and/or other embodiments, the struts may be retracted or extended to accommodate the different size adjustments as described further herein. In addition, the struts may be fixed in the adjusted position through use of retainers (e.g., see, FIG. 6, retainers 190). As previously discussed, in accordance with embodiments the hood 116 may be formed of a material that maintains a given shape without the use of the struts as desired. In further embodiments, the folds 192 may be positioned along a length (e.g., from top to bottom) of the hood 116 to enable modification of an outward extension of the cavity 129. It is further envisioned that more or less folds 192 may be provided to accommodate more or less adjustment, respectively, to the size of the hood 116 as desired.

The opening 128 of the hood 116 may lead to the cavity 129 which may be defined, at least in part, by the at least one wall 118. Attachment openings 135 may extend through an adjacent part of the at least one wall 118 and may include a reinforcement such as grommet or a reinforcement material laminated or otherwise attached to the at least one wall 118 and may be configured to receive corresponding hood couplers (e.g., see, FIG. 1, couplers 126) for coupling to an adjacent support strut 114. As previously described, the openings 135 are sealed from the cavity 129 to ensure that there is no entrance of ambient air into the cavity 129 and no exit of airflow from the cavity 129 to the ambient environment. Further, as described, the openings 135 may constitute a pocket, sleeve, strap, etc., of the hood 116 that may fit around the support struts 114 to slidably receive the support struts 114 in which case, the couplers 126 would not be required.

With particular reference to FIG. 6, the tube 127 may extend away from either side of the base 112 such that one end may be configured to be coupled to the return line 132 and the other end (e.g. on the opposing side) may include the flange 121 and may be configure to receive the opening 123 situated in the top wall 118-T of the hood 116. In contrast and as previously described, the top wall 118-T may extend up to the base 112 and may be secured thereto. In these embodiments, the flange 121 may form a portion of the base 112 with the flange 125 and the base 112 fluidically configured to fluidically seal the top wall 118-T to the tube 127 and thereby, to the return line 132, when each is attached. In either embodiments, the collar 125 may then be coupled to the tube 127 using any suitable method such as a threaded coupler and may secure the top wall 118-T of the hood 116 against the flange 121 to form a suitable seal to prevent or reduce air leakage about the opening 123. In some embodiments, a biasing member such as a spring 141 may be operative to urge the flange 121 (which may be a collar) towards the collar 125 to maintain pressure upon the top wall 118-T situated between the collar 125 and the flange 121 to maintain a seal therebetween. In some embodiments, the tube 127 may be formed integrally with, or separately from, the base 112.

In some embodiments, a sensor, for example positioned on the hood 116 (e.g., within the cavity 129 as discussed), the coupling or otherwise positioned within the flow of air to sense characteristics of the air drawn into/out of the cavity 129 or otherwise present within the airflow. For example, the sensor may sense a CO₂ level and provide such sensor information to the controller to enable a determination by the controller of an increase in the CO₂ level within the airflow. In response, the controller may receive the sensor information and compare a current CO₂ level within the airflow to a desired range of acceptable CO₂ levels and/or to determine that it is below an acceptable (predetermined) level for the airflow. Further, when the current CO₂ level within the airflow is outside the desired range of acceptable CO₂ levels or is higher than the predetermined maximum desired level, the controller may increase the airflow to draw ambient air into the cavity 129 to decrease the CO₂ level within the airflow to a desired acceptable CO₂ level. Further, the controller may control a CO₂ capture system and/or CO₂ venting system as described to reduce the CO₂ level within the airflow to a desired acceptable CO₂ level. By controlling the level of CO₂ within the airflow to a desired CO₂ level, the present system ensures that the airflow provided to the patient is safe for its intended purpose without requiring a supplemental oxygen source be coupled to the system. However, in accordance with embodiments, a supplemental gas (e.g., oxygen source) may be supplied and/or controlled in response to the sensor information (e.g., received from an oxygen or carbon dioxide sensor) to alter the supplied airflow (e.g., oxygen enrich from an oxygen source) as desired. As previously discussed, the sensor may also or alternatively be a pressure sensor to ensure that a negative pressure (e.g., a pressure below ambient pressure) is maintained within the cavity to ensure that no air expelled by the patient is released to the ambient environment without being sterilized by the sterilizer. In addition, other sensors discussed may also be provided inside the cavity of the hood and/or otherwise within the airflow to sense and/or control other characteristics of the air within the airflow.

Support struts 114 may be situated apart from each other and may be coupled to the base 112 using any suitable couple such as a threaded fit, a fastener, an epoxy, an interference fit, etc. For example, in some embodiments, the support struts 114 may pass through corresponding openings 135 (e.g., a portion of the wall 118 sealed from the cavity 129 as described) and may be secured as positioned using any suitable method such as retainers 190, an interference fit or the like. In some embodiments, the proximal end of the support strut may pass fully or partially through corresponding ones of the openings 135. In embodiments wherein the downward extension of the hood 116 is adjustable, such as to adjust a size (e.g., small, medium, large, etc.) and/or to enable an unsymmetrical downward extension to accommodate patients in different orientations (e.g., reclining verses sitting up), the support struts 114 may be extended up above the base 112 to maintain the adjusted position of the hood 116. In these embodiments, the support struts 114 may be maintained in the adjusted position through use of an interference fit or threaded fit through the base 112 and/or through use of the retainers 190. In other embodiments, the support struts 114 may be provided having different lengths and/or adjustable lengths (e.g., break-off tabs) to accommodate different downward extensions of the hood 116, such as when the hood 116 is folded up along one or more of the folds 192. The rear view and the side views of the base 112 may be similar and are not shown for the sake of clarity.

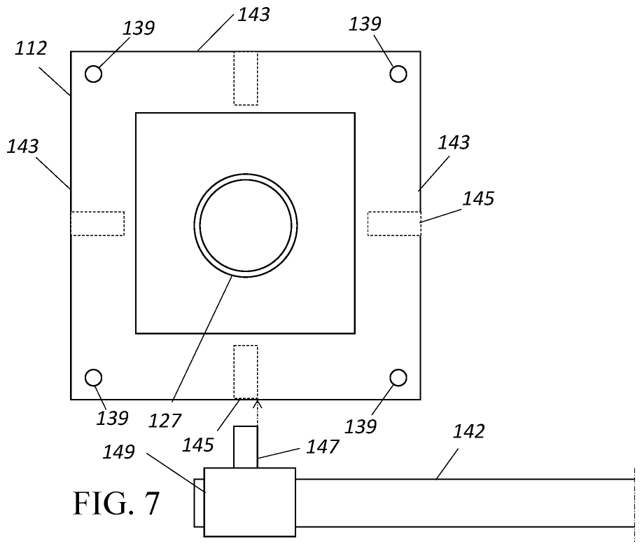

The base 112 may include a coupler configured to couple the base to the support arm (e.g., see second support arm 142, FIG. 1) of the hood support 146. For example, one or more side walls 143 of the base 112 may include one or more openings 145 configured to receive a corresponding coupler that may be coupled to the second support arm as will now be illustratively discussed with reference to FIG. 7.

FIG. 7 is a partially exploded top view which shows a portion of the base 112 in accordance with embodiments of the present system. The one or more openings 145 may be configured to be coupled to a corresponding coupler such as a pin 147 coupled to the support arm 142 via a coupler 149. A fastening mechanism may secure the pin 147 within the corresponding opening 145 of the base 112 to support the base 112 in a desired position relative to the second arm 142. For example, it is envisioned that the fastening mechanism may include a biased latch which may secure the pin 147 in one or more positions that may be continuous or discrete (e.g., for horizontal and vertical positioning, etc.). The biased latch may be depressed by a user to change positions of the base 112 and/or to remove the base 112 completely as may desired. In some embodiments, the pin 147 may be fixedly secured (e.g., with an adhesive, an epoxy, a further lock pin, etc.) within the corresponding opening 145.

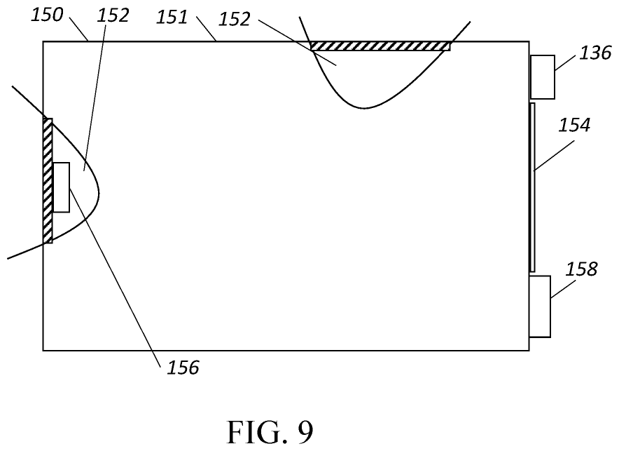

FIG. 8 is a partially-cutaway schematic front view illustration of a portion of the sanitizer 150 in accordance with embodiments of the present system; and FIG. 9 is a partially-cutaway schematic side view illustration of a portion of the sanitizer 150 in accordance with embodiments of the present system. The other side view may be similar and is not shown for the sake of clarity. With reference to FIGS. 8 and 9, the body 151 may include one or more walls which may define at least a portion of the at least one cavity 152. The inlet and outlet ports 136, and 158, respectively, may be flow coupled to the at least one cavity. The air pump such as a blower 159, illustratively shown positioned within the sanitizer 150, may be coupled to the at least one sterilization chamber 152 and may be configured to pump gasses from the at least one sterilization chamber 152 via the outlet port 136 to exhaust gasses from the at least one sterilization chamber 152. In accordance with embodiments, the blower 159 may be positioned at another position within and/or outside the at least one sterilization chamber 152 to control the airflow. When the blower 159 is positioned within the sanitizer 150 and/or at another location, gasses may be drawn in through the inlet port 136 to the at least one sterilization chamber 152.

A controller may control the overall operation of the blower 159 and the STS 156. For example, in some embodiments, if the blower speed is increased, illumination intensity of the STS 156 may also be increased by the controller and/or vice versa. This may assure proper sterilization of gasses such as air within the at least one sterilization chamber 152 regardless of the speed of the blower 159. A user may observe operation of the STS 156 through the window 154. Accordingly, the window may be configured to shield the user from emitted radiation such as UV-C light, etc. In some embodiments, the one or more walls may include a door configured to allow access to the at least one sterilization chamber 152, the blower 159, internal circuitry, and/or the STS 156 for cleaning, repair, and/or replacement of parts of the sterilizer 150. Sealing may be provided around the door to reduce or entirely prevent air leakage. In some embodiments, the STS 156 may include a lighting source, such as an array of LEDs, a plurality of photo tubes, or other illumination sources such as a plurality of illumination sources (e.g., a plurality of arrays of LEDs, etc.). For example, it is envisioned that the STS 156 may include a plurality of sources that may be coupled to one or more of the one or more walls. In some embodiments, the blower 159 may maintain a vacuum within the at least one sterilization chamber 152. A user interface, such as a touchscreen display 157, may be provided for the user to interact with the system. A controller may be coupled to the sterilizer 150 and may communicate with one or more other controllers such as a controller of the ventilator.

By controlling the air pump to pump more air to increase vacuum within the hood, the system may reduce the risk to others (e.g., other than the patient) that occurs, such as when intubated patients must have fluid extracted from their lungs, have respiratory tubes extracted from their internal airways, during eating/drinking, mask adjustment, etc. This may reduce the risk of contamination to the user (e.g., caregivers) and the facility. It is envisioned that embodiments of the present system may reduce or entirely prevent this by maintaining a partial vacuum or negative pressure zone around the head and/or immediately above the head of the patient thereby ensuring that any air and/or water droplets emanating from the patient may be pulled into the hood. During such procedures, a controller of the system may control the air pump to increase flow such that vacuum within the hood (e.g., pressure below ambient pressure) may be temporarily increased to further improve the capacity of the hood to capture air escaping from around the head of the patient. Accordingly, the system may provide a user interface with which a user may select vacuum settings or may select a given procedure (e.g., intubation, extubation, feeding, etc.) in which the controller may control the air pump 159 and/or the STS 156 according to preset settings for the desired procedure or request. In some embodiments, a dial may be provided for a user to set the air flow (e.g., high, med, low) as may be desired.

In some embodiments, one or more sensors 194 may be secured within the chamber 152. In these embodiments, the sensor 194 may be a photon and/or other light sensor coupled to a processor and positioned to sense the light output by the STS 156. In this way, the sensor 194 may determine whether or not the STS 156 is emitting a desired illumination (e.g., wavelength and/or intensity), such as a wavelength that lies in the UV-C spectrum (e.g., 100-280 nm) although other wavelengths of light (e.g., 100-320 nm) are contemplated. In a case wherein the STS 156 is determined to not be producing the desired illumination and the illumination of the STS 156 is adjustable, the controller may attempt to adjust the illumination of the STS 156 to produce the desired illumination as determined by the sensor 194. In a case wherein the STS 156 fails to be adjusted to the desired illumination as determined by the sensor 194 or the STS 156 is not adjustable and is not producing the desired illumination, the controller may cause a user interface of the system to render a message (e.g., a visual and/or auditory rendering) to replace the STS 156 and/or the sensor 194. Further, upon replacement of the STS 156 and/or the sensor 194, the controller may enter a setup operation automatically or by manual selection to ensure that the replacement STS/sensor is operating within desired parameters.

In embodiments of the present system, the sensor 194 may be an ozone sensor to sense ozone within the airflow. As understood, ozone may be created from UV-C light (e.g., from 100 nm to 380 nm, such as 100 nm-110 nm) splitting the 02 molecules in the airflow, thereby creating ozone when the oxygen molecules recombine to make ozone (03). As it is not desired to introduce a significant amount of ozone into the airflow, the sensor 194 may sense the amount of ozone present in the airflow or otherwise produced by the STS 156 and the controller may control the STS 156 (adjust the wavelength of light output by the STS 156) to reduce the amount of ozone produced/present. In a case wherein the STS 156 with adjustment fails to reduce the amount of ozone produced or the STS 156 is not adjustable and is producing ozone above a maximum threshold as determined by the sensor 194, the controller may cause a user interface of the system to render a message (e.g., a visual and/or auditory rendering) to replace the STS 156 and/or the sensor 194. Further, upon replacement of the STS/sensor, the controller may enter a setup operation automatically or by manual selection to ensure that the replacement STS/sensor is operating within desired parameters.

In addition, since it is appreciated that the STS 156 and/or the sensor 194 may degrade over time as may other sensors of the system (e.g., oxygen sensor, ozone sensor, light sensor, etc.), the degradation may be detected (e.g., illumination and/or ozone production) and the controller may provide an indication of the amount of degradation on the user interface of the system. For example, when the STS 156 is determined to be operating well within operating parameters (e.g., plus or minus 10% of an optimal setting), the controller may produce a “green light” indication and/or other equivalent indication so that a user would know that the STS/sensor is in good condition. As the STS 156 is determined to exceed 10% of the optimal setting (e.g., from 10-15% of the optimal setting), the controller may produce a “yellow light” indication and/or other equivalent indication so that a user would know that the STS/sensor is in fair condition and may need to be replaced shortly. Thereafter, when the STS 156 is determined to exceed 15% of the optimal setting, the controller may produce a “red light” indication and/or other equivalent indication so that a user would know that the STS/sensor is in poor condition and needs to be replaced shortly or immediately. Naturally, other ranges of operation may be readily applied and other sensor readings may also be indicated by the provided indication system (e.g., green, yellow and red lights may be applied for CO₂, oxygen, ozone, etc., sensor outputs).

FIG. 10 shows a block diagram of a portion of a system 1000 (hereinafter system 1000 unless the context indicates otherwise) in accordance with embodiments of the present system. The system 1000 may include one or more of: a controller 1010, one or more sensors 1014, a user input interface 1016, a user interface (UI) 1018, a memory 1022, actuators 1024, a ventilator 1028, an airflow circuit (e.g., a patient airflow circuit) 1032, a sterilizer 1030, a patient interface 1034, a network 1040, and a user station (US) 1038, each of which may be coupled to and/or communicate with each other using any communication method or methods such as wired, optical, flow, and/or wireless communication methods. The system 1000 may be operative under the control of the controller 1010. The US 1018 may include any suitable device such as a display, a laptop, a desktop computer, a smart phone or the like and/or other device that may be configured to communicate with other portions of the system 1000 such as the controller 1010 via any suitable communication method or methods such as via the network 1040.

The controller 1010 may include one or more logic devices such as a microprocessor (g) 1012 and may control the overall operation of the system 1000. It should be appreciated that in some embodiments the controller 1050 may include digital and/or analog control circuitry.

It is envisioned that one or more portions of the system 1000 such as the controller 1010 may be operationally coupled to the memory 1022, the user interface (UI) 1018 including a rendering device such as the display 1020, the one or more sensors 1014, and the user input interface 1016, the actuators 1024, the ventilator 1028, the sterilizer 1030, the airflow circuit 1032, the patient interface 1034, the network 1040, and the US 1038.

The memory 1022 may be any type of device for storing application data as well as other data related to the described operation such as application data, SSI, electronic health records (EHRs), electronic medical records (EMRs), operating parameters, clinical decision support (CDS) tools, etc. The application data, SSI, operating parameters, CDS tools, etc., may be received by the controller 1010 for configuring (e.g., programming) the controller 1010 to perform operation acts in accordance with the present system. The controller 1010 so configured becomes a special purpose machine particularly suited for performing in accordance with embodiments of the present system.

The controller 1010 may render content, such as status lights (e.g., green, yellow and/or red lights), still images or video information, on a rendering device of the system such as on the display 1020 of the UI 1018. This information may include information related to operating parameters, system conditions (e.g., STS, sensor, etc.), instructions, feedback, and/or other information related to an operation of the system or portions thereof such as SSI or portions thereof, cluster information, CDS tools, etc. Where SSI may be system setting information that may be used by the system so set operational parameters and settings as may be set by the system and/or user.

The one or more sensors 1014 may be situated at one or more portions of the system and may sense related parameters, form corresponding sensor information, and provide this sensor information to the controller 1010 for further processing. For example, the sensors 1014 may include sensors such as an airflow sensor, a pressure sensor, a CO₂ sensor, an NO₂ sensor, a temperature sensor, a humidity sensor, etc. (as discussed), which may form corresponding sensor information (e.g., airflow, etc.) and provide this information to the controller 1010 for further analysis and/or control of conditions of the airflow. The one or more sensors 1014 may be centrally located and/or may be distributed throughout the system as discussed.

The user input interface 1016 may include a keyboard, a mouse, a trackball, or other device, such as a touch-sensitive display, which may be stand alone or part of a system, such as part of a laptop, a personal digital assistant (PDA), a mobile phone (e.g., a smart phone), a smart watch, an e-reader, a monitor, a smart or dumb terminal or other device for communicating with the controller 1010 via any operable link such as a wired and/or wireless communication link. The user input interface 1016 may be operable for interacting with the controller 1010 including enabling interaction within a UI 1018 as described herein. Clearly one or more of the controller 1010, the sensors 1014, the user input interface 1016, the user interface (UI) 1018, the memory 1022, the actuators 1024, the airflow circuit 1032, the patient interface 1034, the sterilizer 1030, the network 1040, and the US 1038 may all or partly be a portion of a computer system or other device. The UI 1018 may be operative to provide audio/visual feedback to the user of the present system and may inform the operator of operating parameters, operating states, etc.

The methods of the present system are particularly suited to be carried out by a computer software program, such program containing modules corresponding to one or more of: the individual steps or acts described and/or envisioned by the present system. Such program may of course be embodied in a computer-readable medium, such as an integrated chip, a peripheral device or memory, such as the memory 1024 or other memory coupled to the controller 1010.

The program and/or program portions contained in the memory 1022 may configure the controller 1010 to implement the methods, operational acts, and functions disclosed herein. The memories may be distributed, for example between the clients and/or servers, or local, and the controller 1010, where additional processors may be provided, may also be distributed or may be singular. The memories may be implemented as electrical, magnetic or optical memory, or any combination of these or other types of storage devices. Moreover, the term “memory” should be construed broadly enough to encompass any information able to be read from or written to an address in an addressable space accessible by the μP 1012 of the controller 1010. With this definition, information accessible through a network such as the network 1040 is still within the memory, for instance, because the processor 1012 may retrieve the information from the network 1040 for operation in accordance with embodiments of the present system.

The controller 1010 is operable for providing control signals and/or performing operations in response to input signals from the user input device 1016 as well as in response to other devices of a network, such as the one or more sensors 1014 and executing instructions stored in the memory 1022. The μP 1012 may include one or more of: a microprocessor, an application-specific and/or general-use integrated circuit(s), a logic device, etc. Further, the g 1012 may be a dedicated processor for performing in accordance with the present system and/or may be a general-purpose processor and/or circuit wherein only one of many functions operates for performing in accordance with the present system. The μP 1012 may operate utilizing a program portion, multiple program segments, and/or may be a hardware device utilizing a dedicated or multi-purpose integrated circuit.

The actuators 1024 may, under control of the controller 1010, control one or more valves, pumps, and/or motors of the system under the control of the controller 1010. For example, the actuators 1024 may include one or more valve actuators that may control the pressure and/or flow of a fluid such as air used by the system such as in the airflow circuit 1032, a hood (e.g., a negative pressure hood 116), the sterilizer 1030, a CO₂ capture/venting system, an oxygen source, etc.

The ventilator 1028 may include one or more valves, fans, oxygen sources and/or pumps that may be configured to supply a flow of air suitable for breathing by the patient and/or for performing any suitable therapy and as such may include ventilators, respirators, PAP machines, CPAP machines, or other suitable patient gas supply devices, in accordance with embodiments of the present system.

The sterilizer 1030 may include one or more valves, fans, pumps, sensors, radiation sources such as an illumination source for providing germicidal illumination (e.g., UV-C light) in accordance with embodiments of the present system.

The airflow circuit 1032 may pride airflow to the patient interface 1034 alone or together with the ventilator 1028 and may include any suitable air flow path or paths such as may be provided by one or more tubes, hoses, or the like. The airflow circuit 1032 may further include one or more sensors, filters, and/or valves.

The patient interface 1034 may include any suitable patient interface such as a mask, a trach coupling, a hose and/or mouthpiece configured to be coupled to the airflow circuit 1032. When coupled together, the patient interface 11034 and the airflow circuit 1032 provide airflow to the patient. The patient interface 1034 may form a non-invasive patient interface or may be an invasive interface, such as intubation device. The patient interface 1034 may include a valve such as a one-way valve which may be configured to vent the exhaled gasses from the patient interface 1034 via the outlet tube.

Accordingly, embodiments of the present system provide a low-cost system and method for containing contagion inside a self-sterilizing airway loop. Embodiments of the present system may provide a system that may eliminate the need for conventional complex and destructive device sterilization measures that are currently performed when transferring respirators between patients and settings. In accordance with embodiments of the present system there is provided a hood that is formed from a low-cost material such as paper, plastic and/or other materials and may be disposable, thereby eliminating or greatly reducing the effort and expense of undergoing sterilization after use. In accordance with embodiments of the present system, the UV-C light source may extend the service of filters, such as HEPA filters, thus further reducing operational costs and manpower. The compact and portable nature of embodiments of the present system allows respiratory care to be maintained safely in conventional settings such as at home, in hospitals, nursing homes, care centers, in clinics, health systems, surgical suites, acute care settings, emergency departments, waiting rooms, during obstetrics procedures, in ambulances, in sleep centers and/or any other place where a patient may be monitored, treated, transported, receive respiratory assistance or the like. It is also envisioned that health systems employing embodiments of the present system may not need to undergo expensive HVAC system upgrades to assure an adequate level of cleanliness in the area surrounding individuals receiving care such as respiratory assistance.

In accordance with embodiments of the present system, the airflow system may ensure that contagion emanating from the patient travels in a semi-closed circuit thereby not allowing the escape of any airborne pathogens into the caregiver setting. It should be appreciated that the embodiments of the present system may provide for mobility during use such that a patient may use the system when sitting, resting in bed, being mobilized, being transported or while performing tasks such as walking, exercising, using facilities, etc.

It is envisioned that embodiments of the present system may provide for the sterilization of exhalation air (which may include contagions as well as water droplets) emanating from an individual such as one infected with any respiratory contagion. Embodiments of the present system may include a hood covering or positioned over the head of a subject and an air hose emanating from the hood. The air hose is connected to an UV-C sterilizer that may in turn be connected to an ancillary respiratory device or other blower/vacuum which circulates sterilized air back to the subject via a hose and patient interface. In this way and in accordance with the present system, an airflow loop is created from and back to the subject. It is envisioned that the UV-C sterilizer may include a port to pull contaminated air into the device, an air pump such as a vacuum pump, a UV-C light source emanating electromagnetic radiation in a range that disinfects the airflow and an exit port to allow sterilized air to flow out of the UV-C sterilizer towards the patient.

Further variations, combinations of the present system and/or elements thereof would readily occur to a person of ordinary skill in the art and are encompassed by the following claims.

Finally, the above discussion is intended to be merely illustrative of the present system and should not be construed as limiting the appended claims to any particular embodiment or group of embodiments. Thus, while the present system has been described with reference to exemplary embodiments, it should also be appreciated that numerous modifications and alternative embodiments may be devised by those having ordinary skill in the art including using features that are described with regard to a given embodiment with other envisioned embodiments without departing from the broader and intended spirit and scope of the present system as set forth in the claims that follow. In addition, any section headings included herein are intended to facilitate a review but are not intended to limit the scope of the present system. In addition, the specification and drawings are to be regarded in an illustrative manner and are not intended to limit the scope of the appended claims.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function;
e) any of the disclosed elements may be comprised of hardware portions (e.g., including discrete and integrated electronic circuitry), software portions (e.g., computer programming), and/or any combination thereof;
f) hardware portions may be comprised of one or both of analog and digital portions;
g) any of the disclosed devices, features and/or portions thereof may be combined together or separated into further portions unless specifically stated otherwise;
h) no specific sequence of acts or steps is intended to be required unless specifically indicated; and
i) the term “plurality of” an element includes two or more of the claimed elements and does not imply any particular range of number of elements; that is, a plurality of elements may be as few as two elements and may include an immeasurable number of elements.

What is claimed is:

1. A sterilization system for a ventilator return air flow, comprising:
a hood base (112) having an opening and a coupler configured to receive a return line (132) and a plurality of support struts (114) extending away from the hood base;
a hood (116) having a top wall and at least one side wall (118-F, 118-R, 118-S) extending from a top wall (118-T), the hood being configured to be coupled to the plurality of support struts to form an opening (128) leading to a cavity (129), the cavity being configured to receive at least a portion of a head of a subject (101) receiving respiratory gas from an air source (170) via a mask (103);
a sterilizer (150) coupled to the return line (132) and having at least one sterilization chamber (152) and a sterilization source (156) configured to emit radiation to sterilize air within the at least one chamber; and
an air pump (159) configured to establish an air flow through the opening of the hood (116) and into the at least one sterilization chamber for sterilization to form a sterilized air flow that may be provided to an inlet port (172) of the air source,
wherein the hood is configured to enable ambient air into the airflow while over the portion of the head of the subject.

2. The sterilization system of claim 1, further comprising a pole stand (180) comprising a hood support (146) coupled to the hood base, wherein the pole stand is configured to selectively position the hood in a vertical or a horizontal position.

3. The sterilization system of claim 2, wherein the pole stand (180) further comprises a base (184) having a plurality of wheels (182) and a support pole (181) which extends from the base, wherein the base is configured to be coupled to at least one of the sterilizer and the air source.

4. The sterilization system of claim 1, wherein the sterilization source (156) further comprises an ultra-violet (UV) lamp configured to emit radiation that is germicidal into the at least one sterilization chamber of the sterilizer.

5. The sterilization system of claim 4, wherein the emitted radiation has a wavelength between 100-320 nm.

6. The sterilization system of claim 5, wherein the radiation source comprises at least one light-emitting diode (LED).

7. The sterilization system of claim 1, further comprising at least one controller (1010) configured to control the intensity of the sterilization source and speed of the air pump such that increasing the speed of the air pump increases the intensity of the sterilization source and decreasing the speed of the air pump decreases the intensity of the sterilization source.

8. A sterilization system for a ventilator return air flow, the sterilization system comprising:
a return line (132) configured to provide for airflow between first and second ends;
a hood (116) having a top wall (118-F), at least one side wall (118-F, 118-R, 118-S) extending from a top wall (118-T), and an opening situated in the top wall, wherein the hood is configured to be coupled to a tube (127) that is coupled to an end of the return line; and
couplers (126) configured to couple the at least one side wall of the hood to adjacent ones of a plurality of support struts (114) to configure the hood to form an opening (128) leading to a cavity (129), the cavity being configured to receive at least a portion of a head of a subject (101) receiving respiratory gas from an air source (170) via a mask (103),
wherein the hood is configured to enable ambient air into the airflow while over the portion of the head of the subject.

9. The sterilization system of claim 8, further comprising a hood base (112) configured to be coupled to the plurality of support struts (114) such that each of the support struts extends away from the hood base and each other, the hood base having an opening configured to receive the tube that is coupled to the end of the return line.

10. The sterilization system of claim 8, further comprising a sterilizer (150) having at least one sterilization chamber (152) coupled to the return line (132), wherein the sterilizer is configured to receive the airflow, sterilize the airflow, and output the sterilized airflow via an outlet port (158) to be provided to an inlet port (172) of an air source (170).

11. The sterilization system of claim 10, further comprising at least one sterilization source (156) situated within the at least one sterilization chamber, wherein the at least one sterilization source is configured to emit radiation to sterilize the air flow within the at least one sterilization chamber.

12. The sterilization system of claim 10, further comprising a sterilizer output coupler (160) configured to provide the sterilized air flow to the inlet port of the ventilator, the sterilizer output coupler having a balance valve (166) to selectively bleed at least a portion of the sterilized air flow to an ambient surrounding.

13. The sterilization system of claim 12, wherein the balance valve is configured to be manually adjusted.

14. The sterilization system of claim 11, wherein the at least one sterilization source comprises at least one ultra-violet (UV) light emitting diode (LED) configured to emit germicidal radiation into the at least one sterilization chamber of the sterilizer.

15. The sterilization system of claim 11, further comprising at least one controller (1010) configured to control an intensity of the at least one sterilization source.

16. The sterilization system of claim 11, further comprising an air pump (159) configured to establish the airflow to flow air through the opening of the hood (116) and into the at least one sterilization chamber for exposure to the at least one sterilization source and subsequent output as the sterilized air flow to an inlet port (172) of the ventilator.

17. The sterilization system of claim 16, further comprising at least one controller (1010) configured to control the air pump to establish air flow through the opening of the hood.

18. A hood for a sterilization system, comprising:
a return line (132) configured to provide for airflow between first and second ends;
a hood (116) having a top wall (118-F), at least one side wall (118-F, 118-R, 118-S) extending from a top wall (118-T), and an opening situated in the top wall and configured to be coupled to a tube (127) coupled to an end of the return line; and
couplers (126) configured to couple the at least one side wall of the hood to adjacent ones of a plurality of support struts (114) to configure the hood to form an opening (128) leading to a cavity (129), wherein the cavity is configured to receive at least a portion of a head of a subject (101) receiving respiratory gas from a ventilator (170) via a mask (103) such that the plurality of support struts are situated outside of the cavity,
wherein the hood is configured to enable ambient air into the airflow while over the portion of the head of the subject.

19. The hood of claim 18, comprising a sensor configured to sense a CO₂ level within the airflow and to provide sensor information.

20. The hood of claim 19, comprising a controller coupled to the sensor, wherein the controller is configured to reduce the CO₂ level within the airflow.
